(19) United States
(12) Patent Application Publication
(10) Pub. No.: US 2019/0189248 A1
(43) Pub. Date: Jun. 20, 2019

(54) METHODS, SYSTEMS AND APPARATUS FOR SUBPOPULATION DETECTION FROM BIOLOGICAL DATA BASED ON AN INCONSISTENCY MEASURE

(71) Applicant: KONINKLIJKE PHILIPS N.V., Eindhoven (NL)

(72) Inventors: Konstantin VOLYANSKYY, Larchmont, NY (US); Nevenka DIMITROVA, Pelham Manor, NY (US); Yong MAO, Hawthorne, NY (US)

(73) Assignee: Koninklijke Philips N.V., Eindhoven (NL)

(21) Appl. No.: 16/301,060

(22) PCT Filed: May 11, 2017
(86) PCT No.: PCT/EP2017/061248

§ 371 Date: Nov. 13, 2018

Related U.S. Application Data
(60) Provisional application No. 62/338,551, filed on May 19, 2016.

(51) Int. Cl. G16B 40/00

(52) U.S. Cl. CPC G16B 40/00

(57) ABSTRACT

Methods, systems and apparatus for detecting subpopulations of constituents of at least one biological organism are disclosed. In accordance with exemplary embodiments, cluster partitions of biological data samples compiled from constituents of at least one biological organism are evaluated (114) by computing inconsistency scores for the partitions based on an inconsistency measure. In addition, for at least one of the plurality of partitions, a non-zero value is allocated to the inconsistency measure of at least one cluster that has only one biological data sample. Further, the subpopulations are identified by selecting the partition of having the minimum inconsistency score as the subpopulations.

### TECHNICAL FIELD

Various embodiments described herein are directed generally to biomedical informatics technology. More particularly, but not exclusively, various methods, systems and apparatus disclosed herein relate to bioinformatics and detection of subpopulations based on biological data.

### BACKGROUND

Bioinformatics technology provides an efficient means for analyzing biological organisms and is an important aspect of several biological fields. In particular, bioinformatics technological processes have led to significant advancements in genomics and the study and treatment of diseases, including cancer. Cancer, as well as other genome diseases, is characterized by heterogenic patterns of genomic structural variations and gene expression underpinning the evolution from normal to tumor cells. For purposes of clinical studies and, particularly, identification of driver and passenger events in tumor development and proliferation, the ability to interpret and characterize distinctive patterns from available genomic data gains high importance.

### SUMMARY

The effectiveness of currently available biomedical informatics and bioinformatics technologies is relatively limited because the analyses employed by these technologies do not provide a definitive and accurate means for determining the number of subgroups or subpopulations in biological data. For example, the complexity and volume of genetic profiles renders it very difficult to efficiently and accurately analyze them for purposes of detecting various subpopulations including, for example, homogeneous subgroups of cancer patients based on analysis of whole tumor biopsies as well as the clonal populations reflecting a tumor cell lineage and evolution, and populations of abnormal, normal and disease-specific cell lines.

The present disclosure is directed to methods, systems and apparatus for detecting subpopulations of constituents of at least one biological organism. Application of machine learning techniques to discover these types of subpopulations is problematic because the number of classes within the data is often unknown. While non-parametric unsupervised machine learning methods are very good at detecting closeness of individual samples and determining the structure of major subgroups (clusters), they fail to provide a clear indication of the correct number of classes, and parametric methods assume that the number of classes is known in advance, which is rarely the case.

To improve the efficiency of detection of the subpopulations while maintaining a high degree of accuracy, clustering procedures can be performed on the biological data to obtain cluster partitions that are evaluated with an intra-cluster inconsistency measure, such as, for example, a pairwise statistical variance of elements within a cluster. In particular, rather than deeming one-element clusters to have zero inconsistency within the cluster, embodiments of the present application assign a non-zero inconsistency measure to one-element clusters. The inventors of the present application have surprisingly found that analyzing cluster consistency and assigning a degree of intra-cluster inconsistency to one-element clusters enables the emergence of a u-shaped curve with a minimum value of an inconsistency score evaluated as a function of partition levels. Here, the partition level corresponding to the minimum value has been found to accurately denote the number of clusters and the subpopulations present in the biological data. Thus, by assigning a non-zero inconsistency measure to one-element clusters, the subpopulations can be detected in a highly efficient and accurate manner.

Generally, in one aspect, an exemplary system is configured to detect subpopulations of constituents of at least one biological organism. Here, the system is includes at least one hardware processor and a non-transitory storage medium. The processor is configured to obtain a plurality of partitions of biological data samples of the constituents of the biological organism(s), and the storage medium is configured to store the plurality of partitions. In addition, each partition of the plurality of partitions defines a respective number of clusters of the biological data samples of the constituents. Further, the processor is configured to compute, for each partition of the plurality of partitions, an inconsistency score for the corresponding partition based on an inconsistency measure that measures intra-cluster inconsistency, where, for at least one of the plurality of partitions, a non-zero value is allocated to the inconsistency measure of at least one cluster that has only one biological data sample. The processor is further configured to determine which partition of the plurality of partitions has a minimum inconsistency score and to identify the subpopulations of the constituents of the biological organism(s) by selecting the partition of the plurality of partitions having the minimum inconsistency score as the subpopulations.

Similarly, in another aspect, an exemplary method is directed to detecting subpopulations of constituents of at least one biological organism. The method is implemented by at least one hardware processor. In accordance with the method, a plurality of partitions of the biological data samples of the constituents of the biological organism(s) is obtained. In addition, each partition of the plurality of partitions defines a respective number of clusters of the biological data samples of the constituents. For each partition of the plurality of partitions, an inconsistency score for the corresponding partition is computed based on an inconsistency measure that measures intra-cluster inconsistency, where, for at least one of the plurality of partitions, a non-zero value is allocated to the inconsistency measure of at least one cluster that has only one biological data sample. Further, the method includes determining which partition of the plurality of partitions has a minimum inconsistency score and identifying the subpopulations by selecting the partition having the minimum inconsistency score as the subpopulations.

According to exemplary embodiments, the biological data includes at least one of genomic data or proteomic data. System, method and apparatus embodiments have been found to be especially advantageous when applied to genomic or proteomic data due to the significant accuracy in identifying subpopulations.

In one exemplary embodiment, the computing further comprises weighting the inconsistency measure of each cluster of at least a subset of clusters in the corresponding partition as a function of a total number of biological data samples in the corresponding cluster and of a total number of biological data samples of the constituents of the biological organism(s). The weighting can provide an advantageous preference to partitions that have a low intra-cluster inconsistency with relatively small numbers of clusters. In a version of the embodiment, the weighting is performed such that the inconsistency measure of the corresponding cluster is directly related to the total number of biological data samples in the corresponding cluster.

In accordance with an exemplary embodiment, the non-zero value is determined by weighting the inconsistency measure of the biological data samples of the constituents of the biological organism(s) as a whole. Thus, one-sample clusters can, for example, be allocated a part of the overall variance of the partition inconsistency measure of the entirety of the biological samples, thereby enabling the formation of a u-shaped curve and a minimum value in an inconsistency score evaluated as a function of partition levels. As noted above, this minimum value can denote the total number of clusters, thereby permitting an accurate and precise determination of subpopulations. In one version of the embodiment, the inconsistency measure of the biological data samples of the constituents is weighted with a total number of biological data samples of the constituents. In addition, in the same or a different version of the embodiment, the weighting is performed such that the non-zero value is inversely related to the total number of biological data samples of the constituents.

Further, according to an exemplary embodiment, the inconsistency measure is a statistical variance of pairwise distances between biological data samples in a given cluster of the corresponding partition. The use of the statistical variance as the inconsistency measure has been found to be significantly accurate with respect to genomic data.

In addition, in exemplary embodiments, a representation of at least one cluster of the selected partition can be displayed. Moreover, the representation can include at least one of clinical or phenotypic annotations to the cluster(s) to aid a clinician in assessing the data. In one version of the embodiment, the annotations include at least one of drug response data, risk of recurrence of a disease or disease subtype data.

Exemplary embodiments can further include providing diagnostic information. For example, in accordance with one method, at least a subset of clusters of the selected partition is associated with at least one of clinical variables, clinical outcomes or clinical labels. In addition, the method includes receiving at least one biological data sample and searching for at least one match to the biological data sample by comparing the sample to representations of clusters of the selected partition. Moreover, any one or more of clinical variables, clinical outcomes or clinical labels associated with a representation of at least one of the clusters matching the sample is output as diagnostic information. Here, the diagnostic information can serve as a guide for a health care provider in diagnosing or prescribing a particular treatment to a patient. For example, the diagnostic information can indicate a particular cancer subtype from which a patient may be suffering. In addition, the diagnostic information can indicate that one or more particular drugs was successful or unsuccessful in treating a disease or ailment in patients of a cluster matching the biological data sample. Due to the flexibility and adaptability afforded by the embodiments described herein, a wide variety of diagnostic information can be provided.

Further, in one aspect, a computer-readable medium comprises a computer-readable program that, when executed on a computer, enables the computer to perform any one or more of the methods described herein. For example, the computer-readable program can be configured to detect subpopulations of constituents of at least one biological organism such that, when the program is executed on a computer, the program causes the computer to perform the steps of any one or more of the method embodiments described herein. The computer-readable medium can be a computer-readable storage medium or a computer-readable signal medium. Alternatively or additionally, the computer readable medium can include an update or other portion of the computer-readable program.

As used herein for purposes of the present disclosure, the term “constituents of at least one biological organism” should be understood to include, but is not limited to, cells, cell lines, bacterial cultures, other microorganisms or patients.

The term “biological data” should be understood to include, but is not limited to, genomic data, including, for example, one or more of mutations, genome-wide copy number alterations, gene and/or noncoding RNA expression data, DNA methylation data, histone modifications, DNA binding data (e.g. ChIPseq), and/or RNA binding data, and/or other types of genomic data, proteomic data, including, for example, protein expression data, phosphorylation data, ubiquitination data and/or acetylation data of a biological sample, biomedical data, including clinical data and personal health data including glucose level data, blood pressure data, weight data, body mass index (BMI) data, dietary data, and/or daily calorie intake, in addition to other types of biological data.

In addition, a “partition” should be understood to include one or more clusters.

Further, in the embodiments described herein, an “inconsistency measure” is employed, “non-zero” values are allocated to one-element or one-sample clusters, and a “minimum” value of an inconsistency score is determined and employed to identify subpopulations. However, these terms should be understood to include conversely equivalent terms. For example, if a consistency measure, such as, for example, the inverse of a statistical variance, as opposed to an inconsistency measure were employed, then the finding of a “maximum” value of a “consistency” score to identify subpopulations should be understood as being equivalent to determining or finding a “minimum” value of an “inconsistency” score to identify subpopulations. Similarly, in these conversely equivalent cases, the allocation of values to one-element or one-sample clusters, such as, for example, non-unity values of a consistency measure, should be understood to be equivalent to the allocation of a non-zero value of an inconsistency measure to one-element or one-sample clusters.

The term “controller” is used herein generally to describe various apparatus relating to the operation of computing devices. A controller can be implemented in numerous ways (e.g., such as with dedicated hardware) to perform various functions discussed herein. A “processor” is one example of a controller which employs one or more hardware microprocessors that may be programmed using software (e.g., microcode) to perform various functions discussed herein, or employs dedicated hardware. A controller may be implemented with or without employing a processor, and also may be implemented as a combination of dedicated hardware to perform some functions and a microprocessor (e.g., one or more programmed microprocessors and associated circuitry) to perform other functions. Examples of controller components that may be employed in various embodiments of the present disclosure include, but are not limited to, conventional microprocessors, application specific integrated circuits (ASICs), and field-programmable gate arrays (FPGAs).

The term “module” should be understood to be one or more dedicated hardware processors and/or one or more hardware processors executing software instructions.

In various implementations, a processor or controller may be associated with one or more computer-readable storage mediums (generically referred to herein as “memory,” e.g., volatile and non-volatile computer memory such as RAM, PROM, EPROM, and EEPROM, floppy disks, compact disks, optical disks, magnetic tape, etc.). As used herein, the term “non-transitory machine-readable storage medium” will be understood to encompass both volatile and non-volatile memories, but to exclude transitory signals. In some implementations, the storage mediums may be encoded with one or more programs that, when executed on one or more processors and/or controllers, perform at least some of the functions discussed herein. Various storage mediums may be fixed within a processor or controller or may be transportable, such that the one or more programs stored thereon can be loaded into a processor or controller so as to implement various aspects discussed herein. The terms “program” or “computer program” are used herein in a generic sense to refer to any type of computer code (e.g., software or microcode) that can be employed to program one or more processors or controllers. In some implementations, computer readable signal mediums may be encoded with one or more programs that, when executed on one or more processors and/or controllers, perform at least some of the functions discussed herein. For example, a signal medium can be an electromagnetic medium, such as a radio frequency medium, and/or an optical medium, through which a data signal is propagated.

The term “addressable” is used herein to refer to a device (e.g., a controller or processor) that is configured to receive information (e.g., data) intended for multiple devices, including itself, and to selectively respond to particular information intended for it. The term “addressable” often is used in connection with a networked environment (or a “network,” discussed further below), in which multiple devices are coupled together via some communications medium or media.

In one network implementation, one or more devices coupled to a network may serve as a controller for one or more other devices coupled to the network (e.g., in a master/slave relationship). In another implementation, a networked environment may include one or more dedicated controllers that are configured to control one or more of the devices coupled to the network. Generally, multiple devices coupled to the network each may have access to data that is present on the communications medium or media; however, a given device may be “addressable” in that it is configured to selectively exchange data with (i.e., receive data from and/or transmit data to) the network, based, for example, on one or more particular identifiers (e.g., “addresses”) assigned to it.

The term “network” as used herein refers to any interconnection of two or more devices (including controllers or processors) that facilitates the transport of information (e.g. for device control, data storage, data exchange, etc.) between any two or more devices and/or among multiple devices coupled to the network. As should be readily appreciated, various implementations of networks suitable for interconnecting multiple devices may include any of a variety of network topologies and employ any of a variety of communication protocols. Additionally, in various networks according to the present disclosure, any one connection between two devices may represent a dedicated connection between the two systems, or alternatively a non-dedicated connection. In addition to carrying information intended for the two devices, such a non-dedicated connection may carry information not necessarily intended for either of the two devices (e.g., an open network connection). Furthermore, it should be readily appreciated that various networks of devices as discussed herein may employ one or more wireless, wire/cable, and/or fiber optic links to facilitate information transport throughout the network.

The term “user interface” as used herein refers to an interface between a human user or operator and one or more devices that enables communication between the user and the device(s). Examples of user interfaces that may be employed in various implementations of the present disclosure include, but are not limited to, switches, potentiometers, buttons, dials, sliders, a mouse, keyboard, keypad, various types of game controllers (e.g., joysticks), track balls, display screens, various types of graphical user interfaces (GUIs), touch screens, microphones and other types of sensors that may receive some form of human-generated stimulus and generate a signal in response thereto.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter are contemplated as being part of the subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating various principles.

FIG. 1 is a high-level block/flow diagram of a system for detecting subpopulations of constituents of at least one biological organism in accordance with exemplary embodiments.

FIG. 2 is a high-level block/flow diagram of a method for detecting subpopulations of constituents of at least one biological organism in accordance with exemplary embodiments.

FIG. 3 is a diagram illustrating a plot of inconsistency scores that can be employed to identify the subpopulations of constituents of at least one biological organism in accordance with exemplary embodiments.

FIG. 4 is a high-level block/flow diagram of method for providing diagnostic information in accordance with exemplary embodiments.

FIG. 5 is a high-level block/flow diagram of an exemplary computer system that can implement one or more exemplary embodiments.

### DETAILED DESCRIPTION

Within biomedical informatics, bioinformatics analysis of genomic data is generally very difficult due to the complexity and size of the data. In particular, currently available technologies do not provide an adequate means for determining the number of subgroups or subpopulations in biological data. The analysis is especially difficult when it is applied to patient clinical data, personal health data and genomic data from a very large cohort of patients, cell lines and/or cells for purposes of detecting subpopulations, which can include, for example, patient subgroups at a population study level as well as clonal populations of disease cells or different cell-lines associated with a disease. To improve the accuracy and efficiency of detecting subpopulations, applicants have recognized and appreciated that it would be beneficial to assign a non-zero intra-cluster inconsistency measure starting with one-element clusters. Allocating a non-zero intra-cluster inconsistency measure to one-element clusters in this way is counter-intuitive, but enables the emergence of a u-shaped curve with a minimum value in an inconsistency score evaluated as a function of partition levels. This minimum value has been found to accurately denote the correct number of subpopulations in biological data. Thus, by assigning a non-zero intra-cluster inconsistency measure to one-element clusters, exemplary embodiments provide an efficient and elegant means for identifying the subpopulations.

The identification of subpopulations as described herein can be employed as a diagnostic tool. For example, the identification of subgroups/subpopulations can be employed in distinguishing subpopulations of patients with similar patient characteristics and similar outcomes. In addition, identification of subpopulations can be employed in clinical applications for purposes of discerning patterns of clonal evolution and tumor heterogeneity in assessments of aggressiveness of the tumor sample. This insight provides significant advantages in the treatment of cancer, as well as other diseases. Thus, embodiments can be employed to aid in the treatment planning phase of the patient journey. For example, the embodiments can be utilized in therapy design based on diagnosis at the cell population level. Here, the identification of subpopulations is particularly advantageous, as doctors can tailor drugs and inhibitors to each subpopulation, rather than using one inhibitor on an average target. Thus, in this way, certain subpopulations that are shown by embodiments to be particularly aggressive can be specifically targeted to treat a patient. Embodiments described herein can also be used to discover new population outgrowth in bacterial infections and can be used to distinguish between hospital-acquired (nosocomial) infections and community acquired infections.

In view of the foregoing, various embodiments and implementations described herein are directed to methods, systems and apparatus for detecting subpopulations of constituents of at least one biological organism. The embodiments can be employed to, for example, classify genomic and/or transcriptomic events, characterize clonal cell populations, and extract valuable clinical information, such as tumor progression patterns, prognosis of treatment plan efficacy, and patient risk. Further, embodiments can include a pattern recognition tool that can detect clonal populations based on genomic data including, for example, mutations, genome-wide copy number alterations, gene and/or noncoding RNA expression data, DNA methylation data, histone modifications, DNA binding data (e.g. ChIPseq), and/or RNA binding data, in addition to other types of genomic and proteomic and post-translational modifications data. In accordance with exemplary aspects, clonal populations can be detected from proteomic data that are extracted from mass spectrometry methods and can be incorporated into the integrated analysis. Mertins et al., “Integrated proteomic analysis of post-translational modifications by serial enrichment,” Nature Methods 10, 634-637 (2013), incorporated herein by reference, describes an example of a mass spectrometry method. The proteomic data can include protein expression data, phosphorylation data, ubiquitination data and acetylation data of a biological sample. Moreover, in accordance with exemplary embodiments, intra- and inter-cell heterogeneity can be characterized in automated fashion for purposes of genome disease studies and patient clinical assessment. In addition, the embodiments can also detect subpopulations in bacterial evolution for infectious disease management and antibiotic resistance detection and prediction.

Exemplary method and system embodiments can identify patterns in various types of genomic/proteomic data in a combined or separate fashion to characterize patient data for clinical outcome prediction and subtyping. As indicated above, preferred embodiments can integrate and extract useful information from available modalities of genomic and/or proteomic information. Further, exemplary system and method embodiments can be implemented as an efficient computational tool for genomic pattern recognition using a multi-level clustering architecture and for data interpretation in a clinical context.

Moreover, exemplary embodiments can be employed to determine subpopulations within a large group of organisms (individuals) with a certain level of heterogeneity of overall characteristics measured by different technologies from the medical-clinical perspective, including data from electronic medical records, physiological signals, and/or health data. For example, the embodiments can be employed to classify patients based on disease information (e.g. tumor grade, nodular involvement, stage, metastasis status, immunohistochemistry status, age, drug response data, overall survival and progression free survival data etc.), continuous health data (e.g. heart rate, number of steps per day, deep and shallow sleep patterns, galvanic skin response measurements), etc.

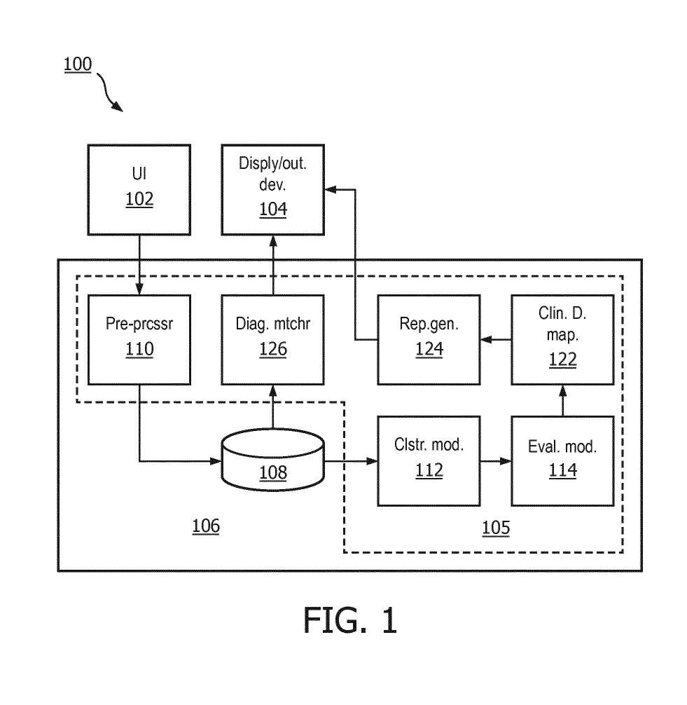

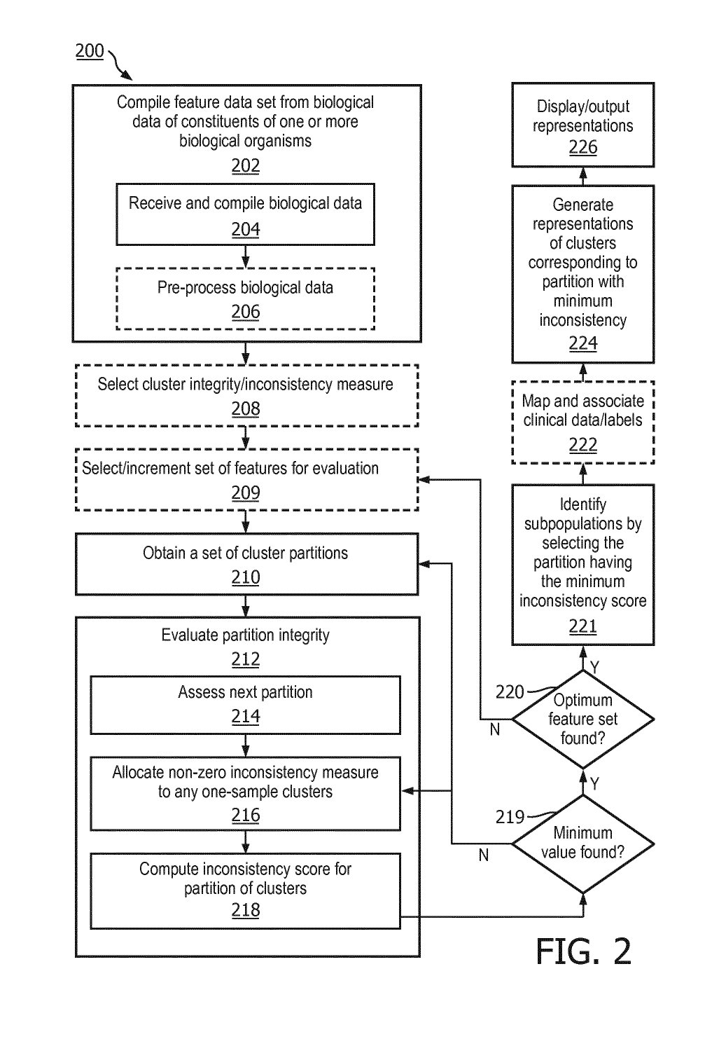

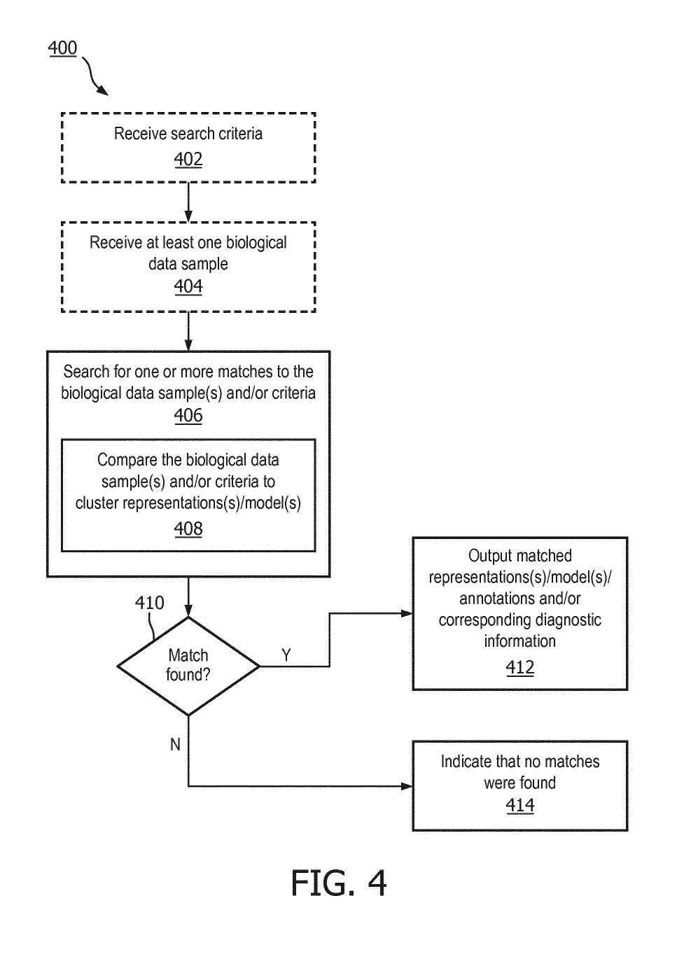

With reference to FIG. 1, an exemplary system 100 for detecting patterns and/or subpopulations of constituents of at least one biological organism in accordance with exemplary embodiments is illustratively depicted. The system 100 can include a pre-processor (Pre-prcssr) 110, a cluster module (Clstr. Mod.) 112, a partition evaluation module (Eval. Mod.) 114, a clinical data mapper (Clin. D. Map.) 122, a representation generator (Rep. Gen.) 124, and a diagnostic matcher (Diag. Mtchr) 126. Each of the system components 110, 112, 114, 122, 124 and 126 can be implemented by a controller (Cntrlr) 105, which can be one or more hardware processors that are part of a hardware computing system 106. The computing system 106 can also include a storage medium 108, and the system 100 can include a user-interface (UI) 102 and a display/output device (Dsply/Out. Dev.) 104. In some embodiments, the user-interface 102 and the display/output device 104 can be incorporated into a single device, such as, for example a touch-screen device. Exemplary functions of the various system components in accordance with exemplary embodiments are described herein below with respect to the method 200 of FIG. 2 and the method 400 of FIG. 4.

Referring to FIG. 2, with continuing reference to FIG. 1, an exemplary method 200 for detecting subpopulations of constituents of at least one biological organism is illustratively depicted. Here, the constituents can be cells, e.g., clonal cells, or cell lines of one or more organisms. Alternatively or additionally, the constituents can be the biological organisms themselves, including, for example, patients or even bacterial cultures. The method 200 can be applied to detect subpopulations of any one or more of these constituents based on biological data, including, for example, genomic data and/or proteomic data, compiled from the constituents. It should be noted that the methods 200 and 400 can be performed by the system 100 or 106. For example, the steps of the method 200, and the method 400, can be instructions of a program that can be stored on the storage medium 108 and executed by a controller implementing elements 110, 112, 114, 122, 124 and/or 126, as, for example, discussed herein below.

The method 200 can begin at step 202, at which the pre-processor 110 can compile a feature data set from biological data samples of constituents of one or more biological organisms. For example, the pre-processor 110 can receive the biological data samples at step 204 and, in one embodiment, can directly compile the data in one or more matrices. The input data can also be received in the form of a data matrix or a set of data matrices, which can be merged or analyzed separately. For example, the method 200 can be performed for each of the data matrices in a set.

The biological data received and compiled at step 204 can include at least one of genomic data, proteomic data or clinical data. For each member of the cohort, the genomic data can include, as discussed above, one or more of mutations, small insertions and deletions (Indels), rearrangements, genome-wide copy number alterations, gene expression data, methylation data, and/or other types of genomic data. Alternatively or additionally, as noted above, proteomic data can include protein expression data, phosphorylation data, ubiquitination data and/or acetylation data of a biological sample. Proteomic data is the functional readout of the genomic architecture and many downstream biological processes. The genomic and/or proteomic data may be composed of one of the types of data described above or any combination of the different types of data. The copy number alterations can denote deletions and amplifications for various regions of a genome for each member of the cohort. Gene expression data and methylation data represent additional types of genome characterization in terms of over/under expression of genes and degree of gene silencing or activation in a given biological organism respectively. These data are provided as quantitative variables derived from measurement procedures and can be part of the input received at step 202. It should also be noted that although genomic and proteomic data are described here as examples, the biological data can additionally or alternatively include other types of data, as noted above. As understood by those skilled in the art based on the present Specification, the data can be formulated and analyzed in a manner similar to the examples described herein below with respect to genomic data.

It should be noted that, in addition to biological data, the user may optionally input annotations/labels, which can comprise clinical variables, clinical outcomes and/or other clinical labels, to the system 106 at step 202. The annotations/labels are discussed in detail herein below with respect to steps 222 and 224, and also method 400 depicted in FIG. 4.

At step 204, as indicated above, the pre-processor 110 can formulate the biological data samples compiled from the cohort of the constituents of the biological organism(s) as a matrix within at least one data structure of the storage medium 108. Here, each column of the matrix can be a biological data sample of a constituent of the biological organism(s). For example, genomic data compiled from the cohort can be formulated as follows:

$$( 1 )$$

$$\begin{matrix}\text{?} & … & \text{?} & \text{?} & … & \text{?} & \text{?} & … & \text{?} \\ {CNA}_{1 , 3} & … & {CNA}_{N , 3} & {GE}_{1 , 3} & … & {GE}_{M , 3} & {M}_{1 , 3} & … & {M}_{N , 3} \\ ⋮ & ⋮ & ⋮ & ⋮ & ⋮ & ⋮ & ⋮ & ⋮ & ⋮ \\ {CNA}_{1 , M} & … & {CNA}_{N , M} & {GE}_{1 , M} & … & {GE}_{M , N} & {M}_{1 , N} & … & {M}_{N , M}\end{matrix}$$

$$\text{?} \text{indicates text missing or illegible when filed}$$

In this particular example, the genomic data consists of copy number alteration (CNA), gene expression data (GE), methylation data (M). However, it should be understood that the matrix can be composed of one of these types of data or any sub-combination of these types of data or other types of data discussed above. In addition, the matrix can include measurements of phenotypic expression, e.g. tumor volume, grade, stage, age, response to a drug, time to progression, and/or time to death if the matrix represents a whole population of organisms. Alternatively or additionally, the matrix can include measurements of individual cells of a biological organism both at the genomic and epigenomic as well as the protein levels. Further, each set of columns denotes a particular member of a cohort, which can be, for example, a particular cell of a given patient, or a particular patient. In addition, as noted above, each column can be a biological data sample. For example, if the cohort members are patients, the patients are denoted by the first subscript in the elements of matrix (1), where CNA_{1,n}, GE_{1,n} and M_{1,n} respectively denote copy number alteration data, genome expression data and methylation data of patient 1, CNA_{2,n}, GE_{2,n} and M_{2,n} respectively denote copy number alteration data, genome expression data and methylation data of patient 2, etc. Here, n denotes an arbitrary chromosome region of a genome, where the genome of each patient in the cohort is delineated by 1, 2, 3 . . . M regions along the genome length. The delineated regions are denoted by the rows in Matrix (1). For example, CNA_{1,1}, GE_{1,1} and M_{1,1} denote copy number alteration data, genome expression data and methylation data, respectively, of region 1 of patient 1, CNA_{1,2}, GE_{1,2} and M_{1,2} denote copy number alteration data, genome expression data and methylation data, respectively, of region 2 of patient 1, CNA_{2,2}, GE_{2,2} and M_{2,2} denote copy number alteration data, genome expression data and methylation data, respectively, of region 2 of patient 2, etc. Thus, CNA_{m,n} can denote a normal alternation, a deletion or an amplification in region m of the genome of patient n, while GE_{m,n} can denote values of genes that are expressed at region m of the genome of patient n. The delineated regions of the genome can also be received at step 202 and subsequently arranged as a column vector, which can be stored in a storage structure within the storage medium 108 and can be a reference employed by any one or more of the elements of the system 106 to map elements of matrix (1) to particular genome regions. Thus, in matrix (1), each column can denote a different patient in the cohort, where the first subscript in any matrix element denotes a particular patient in the cohort, and each region 1, 2, 3 . . . M corresponds to the genomic data for that patient.

It should be noted that, preferably, the method 200 is implemented for one data type. For example, the method 200 can be performed for copy number data, denoted by columns CNA_{n,1}. . . . CNA_{n,M}. In addition, the method 200 can be performed in parallel for gene expression data and methylation data separately. However, it should be understood that the method 200 can be performed on the entire data of the matrix (1) in one implementation of the method. The method 200 can be also applied to other types of data measurements of biological activity of an organism or cells within an organism. Here, genomic level data is analyzed, however, it should be understood that the method is equally applicable to disease information (e.g. tumor grade, nodular involvement, stage, metastasis status, immunohistochemistry status, age etc.), continuous health data (e.g. heart rate, number of steps per day, deep and shallow sleep patterns, galvanic skin response measurements) and therapy response information, including drug response/resistance data, overall survival and progression free survival.

In accordance with one embodiment, the matrix (1) can be the feature data set compiled in step 204. Alternatively, the matrix (1) can be further pre-processed to obtain a feature data set that is analyzed in step 208/210 and subsequent steps. For example, optionally, at step 206, the pre-processor 110 can perform data centering, normalization and/or outlier detection on the data received and compiled at step 204. Here, to perform data centering, the pre-processor 110 can compute and subtract mean values in the feature vectors as follows:

X:=X−M(X) (2)

where X is the feature vector, which can be matrix (1), a column of matrix (1) or a set of columns in matrix (1), and M(X)=Average(X)

Further, to perform data normalization, the pre-processor 110 can employ a transformation that is most appropriate for the specific data type. For example, the pre-processor 110 can implement the normalization by performing one of the following procedures. In accordance with a first procedure, the pre-processor 110 can divide each feature vector by the maximum element as follows:

X:=X/MAX(X) (3)

where X is the feature vector, which can be matrix (1), a column of matrix (1) or a set of columns in matrix (1), and MAX(X) is the maximum element in the feature vector X. In a second procedure, the pre-processor 110 can compute the standard deviation and divide each feature vector by a respective standard deviation as follows:

X:=X/STD(X) (4)

where X is the feature vector, which can be matrix (1), a column of matrix (1) or a set of columns in matrix (1), and STD(X) is the standard deviation of the feature vector X. In accordance with the third procedure, the pre-processor 110 can compute each feature range and can divide the feature vector by the range length as follows:

X:=X/LENGTH(RANGE(X)) (5)

where X is the feature vector, which can be matrix (1), a column of matrix (1) or a set of columns in matrix (1), and RANGE(X) is the range of values in the feature vector seen in a particular sample cohort, and LENGTH(RANGE(X)) is the length of the range.

Further, to perform outlier detection, at optional step 206, the pre-processor 110 can identify outliers in the biological data received at step 204 and can separate the outliers from the biological data. Thus, the feature data set compiled by the pre-processor 110 at step 202 can be the centralized and normalized data set without any identified outliers. For example, to determine and separate outliers, the pre-processor 110 can apply one or more of a variety of approaches, including at least one of a Mahalanobis distance method or a principal component analysis (PCA) method. Here, the pre-processor 110 can apply one of these methods, both of the methods or any sub-combination of these methods with any appropriate method that identifies and separates outliers. For each of these methods, the biological data received at step 204 can be composed in a data matrix.

In the Mahalanobis distance method, the pre-processor 110 can split the data matrix, which would typically have a high dimension, into regions. Here, each data category can be grouped in the matrix, as, for example, adjacent columns. For example, genome-wide copy number alteration data can be grouped in a set of adjacent columns, gene expression data can be grouped in a set of adjacent columns, methylation data can be grouped in a set of adjacent columns, etc. The pre-processor 110 splits the matrix such that each category set is split into multiple regions, so that any given region is composed of data from only one category. For each region and data category, the pre-processor 110 can compute a mean value estimate M(X) and a covariance estimate C(X) as follows:

$$\begin{matrix}M ( X ) = Average ( X ) & ( 6 ) \\ C ( X ) = \frac{1}{n - 1} \sum {( x - M ( X ) )}^{T} ( x - M ( X ) ) & ( 7 )\end{matrix}$$

where X denotes a data category, which can be, for example, a copy number alteration category, a gene expression data category or a methylation data category, x denotes a value or element in the region, and n here denotes the number of elements in the region (n≥2). The pre-processor 110 can compute the Mahalanobis distance MD(x,X) or each element x in quadratic form as follows:

MD(x,X)=(x−M(X))C⁻¹(X)(x−M(X)) (8)

Further, the pre-processor 110 can detect outliers as points with large Mahalanobis, determined as Mahalanobis distances that are above a threshold. The pre-processor 110 can also evaluate the Mahalanobis distances using a chi-squared (χ²) distribution of degrees of freedom identified from the region dimension (n-1).

In the PCA analysis method, the pre-processor 110 can linearly transform (rotate) the original data matrix such that the correlation matrix is diagonalized in the transformed space. Here, the pre-processor 110 can split the correlation matrix into regions, as for example discussed above with regard to the Mahalanobis distance method, and can select the number of principal components based on the threshold of variance captured by these components. For example, the threshold can be chosen to be 90%. The pre-processor 110 can compute the Mahalanobis distance on the obtained principal components as discussed above with respect to equations 6-8 and can apply the chi-squared test to identify abnormally high values as outliers, as discussed above.

In accordance with exemplary embodiments, the preliminary feature data set can be composed of data resulting from step 206, or step 204. After compiling the feature data set at steps 204 and/or 206, the pre-processor 110 can store the feature data set within a data structure in the storage medium 108 for subsequent retrieval by the cluster module 112, or can provide the feature data set directly to the cluster module 112.

Optionally, at step 208, the cluster module 112 can select a cluster integrity measure. For example, the cluster integrity measure can be an inconsistency measure, such as, for example, a variance, which measures an intra-cluster inconsistency. The cluster integrity measure can be a variance of pairwise distances between samples in each cluster/subgroup of a given partition determined by a clustering procedure, which can be performed at step 210. Here, the variance should be understood as a statistical variance measure. For example, the variance can be denoted by

$$VAR ( {C}_{r} ) = \frac{{\sum }_{i , {i}^{′} ∈ {C}_{r}} {( {d}_{i , {i}^{′}} - {d}_{μ} )}^{2}}{K} \qquad ( 9 )$$

where VAR(Cᵣ) is the variance of cluster Cᵣ, d_{i,i′} is the distance between a given pair of samples/constituents i and i′ in the cluster Cᵣ, d_{μ} is the average distance taken over all possible pairs of samples in cluster Cᵣ and K is the the total number of samples/constituents in the cluster Cᵣ. Further, the distance measure d_{i,i′}, d_{μ} can be a Euclidean distance measure, a Manhattan distance measure, or other appropriate distance measure. Alternatively, the cluster integrity measure can be the entropy of the samples/constituents in the cluster Cᵣ. For expository purposes, the variance is used herein below. However, the method 200 can employ other types of cluster integrity measures, where VAR(X) can denote a cluster integrity measure or inconsistency measure in general and can be supplanted as one or more other integrity measures herein below. For example, the user can input and define the cluster integrity measure to be employed at the evaluation step 212, described herein below. Alternatively or additionally, the cluster module 112 can provide a user with several options of cluster integrity measures through the user-interface 102 and the cluster module 112 can select the cluster integrity measure chosen by the user for use at step 212. Alternatively, the cluster integrity measure can be pre-determined and applied by the system 106 in all cases.

Optionally, at step 209, the cluster module 112 can select and/or increment a set of features for evaluation. For example, the method 200 can iteratively assess different sets of genes from, for example, the rows of matrix(1), to determine which set of genes, or features in general, best identify the optimum number of clusters. In accordance with one exemplary embodiment, at step 209, the clustering module 112 can determine the subsets of features, which can be, for example, subsets of rows of matrix (1), having the highest variance. For example, the clustering module 112 can calculate the variance of different sets of features and determine the top 1% of features, or genes in this example, having the highest variance. Similarly, the clustering module 112 can also determine the top 5%, 10%, 15%, etc. of features having the highest variance. Here, in combination with optional step 220, each of these sets of features can be iteratively evaluated by steps 210-219, as discussed herein below. Accordingly, in a first iteration of the loop defined by steps 209 and 220, steps 210-219 can be applied to the set of features corresponding to the top 1% of features having the highest variance. However, it should be understood that steps 209 and 220 are optional and that steps 210-219 can be applied to the feature data set provided by step 202.

At step 210, the cluster module 112 can obtain a set of cluster partitions, where each partition of the set or plurality of partitions defines a respective number of clusters of the biological data samples of the constituents. For example, the cluster module 112 can perform a clustering procedure to generate the set of partitions. Alternatively or additionally, the cluster module 112 can receive the set of cluster partitions of the biological data samples as an input from a user at step 202. For example, for a given set of samples, such as, for example, matrix (1) or a matrix composed of a given subtype of data, e.g., copy number alteration (CNA), gene expression data (GE), methylation data (M), the cluster module 112 can generate or accept as a given input a set of distinct cluster partitions of the input samples. Here, the cluster module 112 can perform an unsupervised cluster procedure such as, for example, hierarchical clustering, fuzzy clustering, k-means clustering, or any other type of clustering scheme. In addition, each partition can define a different number of clusters of the biological data samples. For example, one partition can define one cluster, a second partition can define two clusters, etc.

At step 212, the partition evaluation module 114 can evaluate the partition integrity of the clusters obtained at step 210. For example, for each partition of at least a subset of the partitions obtained at step 210, the partition evaluation module 114 can compute an inconsistency score for the corresponding partition based on an inconsistency measure that measures intra-cluster inconsistency. As noted above, the inconsistency measure can be the statistical pairwise variance within a cluster, or can be an entropy measure of a cluster, for example. In accordance with one exemplary implementation, the partition evaluation can be performed iteratively, where the partition evaluation module 114 assesses the next partition at step 214. For example, the procedure can be initiated with the partition number set to zero and increased to one here at step 214. The partition number can be used to identify a partition and can correspond to the number of clusters defined by the partition. Alternatively, the evaluation module 114 can increment the partition number by values greater than 1 initially and/or subsequently. For example, the partition number incremented at step 214 can be increased and/or decreased throughout the iterative process. However, in the particular implementation described herein below, at each iteration of the step 214, the partition number can be incremented by one. Alternatively, as opposed to increasing the partition number, as discussed above, the evaluation module 114 can decrease the partition number in the same manner discussed above. Indeed, the iteration of step 214 can be implemented in a variety of different ways as long as a sufficient number of partitions are evaluated to decipher a minimum value of the integrity/inconsistency score, as discussed herein below.

At step 216, the partition evaluation module 114 can allocate a non-zero inconsistency measure to any cluster that has only one biological data sample. For example, when the statistical variance is employed as the inconsistency measure, the variance (S_i) of the single-sample cluster can be determined by allocating a part of the overall variance of the partition assessed in the iteration of step 212 to the single-sample cluster. For example, when the statistical variance of equation (9) is employed as the inconsistency measure, the variance of the single-sample cluster, VAR(S_i), can be determined as follows:

$$VAR ( S\_i ) = ( \frac{1}{N} ) VAR ( TotalPartition ) \qquad ( 10 )$$

where N is the total number of the biological data samples of the constituents and VAR(TotalPartition) is the variance of the total partition. In other words, VAR(TotalPartition) is the pairwise variance of all biological data samples of the constituents of the biological organism(s) as a whole. In addition, N here can be, for example, N in matrix (1) for copy number alteration data, gene expression data or methylation data, of the constituents of the biological organism(s). Thus, the partition evaluation module 114 can determine the non-zero value, VAR(S_i), by weighting the inconsistency measure, e.g., VAR(TotalPartition), of the biological data samples of the constituents of the biological organism(s) as a whole with a total number of biological data samples N of the constituents of the at least one biological organism. Thus, in accordance with exemplary aspects, the weighting can be performed such that the non-zero value, VAR(S_i), is inversely related to the total number (N) of biological data samples of the constituents of the biological organism(s). As indicated above, the allocation of non-zero inconsistency measures for one-sample clusters is counterintuitive, but provides a substantial advantage in that it enables the development of a u-shaped plot of the inconsistency scores, thereby permitting an identification of the optimum partition of clusters of the biological data samples.

At step 218, the partition evaluation module 114 can compute an inconsistency score for the corresponding partition under evaluation based on the cluster integrity measure/inconsistency measure. For example, if the inconsistency measure is the pairwise statistical variance, as discussed above, the inconsistency score SCORE_VAR(Partition) for the partition under evaluation at the current iteration of step 218 can be calculated as follows:

SCORE_VAR(Partition)=D₁VAR(C₁)+D₂VAR(C₂)+ . . . +D_{R}VAR(C_{R}) (11)

where C₁, C₂, . . . C_{R} respectively denote clusters 1, 2 . . . R in the partition, and R denotes the total number of clusters in the partition. However, it should be understood that, if any cluster Cᵣ is a one sample cluster, “VARS(S_i)”, for Cᵣ, should replace “DᵣVAR(Cᵣ)” in equation (11). In addition, in accordance with preferred embodiments, the coefficients Dᵣ, where r=1, . . . , R, can be chosen as a function of the number of elements in cluster Cᵣ and of the total number of biological samples. In other words, the coefficients Dᵣ, r=1, . . R, be a function of the total number of biological data samples in the corresponding cluster Cᵣ and of the total number of biological data samples, e.g., N in matrix (1) for copy number alteration data, gene expression data or methylation data, of the constituents of the biological organism(s). Thus, by, for example, applying the coefficients Dᵣ to the inconsistency measure VAR(Cᵣ) in accordance with equation (11), the partition evaluation module 114 can weight the inconsistency measure of each cluster Cᵣ of the clusters in the corresponding partition as a function of a total number of biological data samples in the corresponding cluster and of a total number of biological data samples of the constituents of the at least one biological organism. Configuring the coefficients to be a function of the total number of elements in a cluster and the total number of samples can improve and better define the u-shape of the plot of the inconsistency scores, thereby better enabling the determination of an optimum partition. In accordance with one exemplary implementation, the coefficients Dᵣ can be computed as

$${D}_{r} = \frac{{s}_{r}}{N} ,$$

where sᵣ is the total number of biological data samples in the corresponding cluster Cᵣ, and N is the total number of biological data samples of the constituents of the biological organism(s). Accordingly, the inconsistency measure DᵣVAR(Cᵣ) of the corresponding cluster Cᵣ can be directly related to the total number sᵣ of biological data samples in the corresponding cluster Cᵣ. The direct relation weights and gives advantage or preference to given clusters with a variance VAR that have a larger number of elements sᵣ as compared to other clusters with the same variance VAR that have a smaller number of elements than the given clusters. Thus, the coefficients effectively provide an advantageous weighting to partitions that have a low variance with small numbers of clusters. However, it should be understood that other implementations of the coefficients Dᵣ can be employed in which this same or similar advantage or preference is applied.

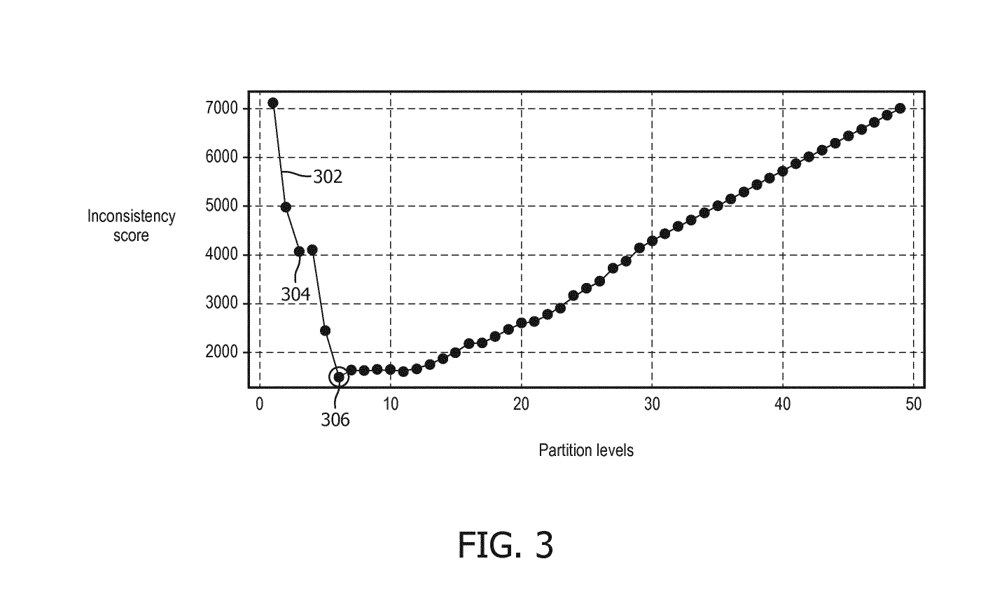

At step 219, the partition evaluation module 114 can determine whether a minimum value of the inconsistency score has been found. For example, the partition evaluation module 114 can compile all of the inconsistency scores of partitions evaluated through iterations of the step 212 and assess the inconsistency scores with respect to the total number of clusters of the evaluated partitions. For example, in accordance with one implementation, the partition evaluation module 114 can form a plot, such as plot 302 of FIG. 3, where the vertical axis denotes the inconsistency score and the horizontal axis denotes the total number of clusters in a given partition, referred to in FIG. 3 as a partition level. Here, each point on the plot 302 denotes the total number of clusters in a different partition and the plot can be constructed as inconsistency scores are determined for the partitions. In accordance with FIG. 3, each partition has a unique number of clusters selected from the range of one cluster to approximately 50 clusters. For example, the clustering procedure, if performed at step 210 or performed separately, can determine the partitions for a large number of partitions, in this example, 50 or more partitions. In addition, the partition evaluation module 114 can iteratively determine the inconsistency scores and iteratively construct the plot until a definitive minimum has been found. For example, at step 219, the partition evaluation module 114 can add the most recently determined inconsistency score to the plot and determine whether a minimum value has been found. The minimum value can be found by assessing the curve in the plot to determine the point in the curve at which the first derivative is zero. Here, the point in the curve at which the first derivative is zero corresponds to the minimum value. If the minimum value has not been found, then the method can proceed to step 212, at which another inconsistency score for the next partition level or number can be determined. The plot can be constructed consecutively from a partition level of one (one total cluster), to a partition level of 2 (two total clusters), etc. until a minimum value has been found. Alternatively, the method can proceed to step 210 if a minimum value has not been found. For example, the partition evaluation module 114 can be configured to determine that a minimum value has been found after 10 or 20 additional inconsistency scores after a lowest value has been added to the plot. For example, this feature would prevent the partition evaluation module 114 from detecting a false positive, as at point 304, in which a local minimum outlier has been found. Here, the partition evaluation module 114 can obtain additional inconsistency scores to find the true minimum value, which in this case is at point 306. The threshold of additional inconsistency scores can be set as a matter of design choice that is based on the features of the particular data examined and that balances processing efficiency with accuracy. Thus, in case a sufficient number of partitions have not been obtained at step 210, or, equivalently, if all available partitions have been assessed and the threshold has not been reached, then the method can proceed to step 210 to obtain additional partitions, by either performing a clustering procedure or obtaining additional partitions from an outside source, such as an outside or remote database.

If, at step 219, the partition evaluation module 114 determines that a minimum inconsistency score has been found, then the method can proceed to step 221, or can proceed to optional step 220. For example, as indicated above, the partition evaluation module 114 can determine that the minimum inconsistency score is the lowest value on the plot when a threshold number of inconsistency scores have been added after this lowest value and no lower value of the inconsistency score has been found. In the example in FIG. 3, the minimum value of the inconsistency score corresponds to point 306, which denotes the partition defining a total of six clusters.

At optional step 220, the cluster module 112 can determine whether an optimum feature set has been found. For example, in the first iteration of the loop defined by steps 209 and 220, the cluster module 112 can assess the set of features corresponding to the top 1% of features having the highest variance. Here, the cluster module 112 can assess the sharpness or steepness of the curve in the plot discussed above about the minimum value. For example, the cluster module 112 can determine the sequence of normalized absolute differences between adjacent points on the variance score curve: Sₙ={100*|VS(N)−VS(n−1)|/(VSₘₐₓ−VSₘᵢₙ)}, n=2, 3, . . . , nₘᵢₙ, where nₘᵢₙ, is the partition having the minimum inconsistency score, VS(n) is the inconsistency score at partition level n, VSₘₐₓ is the maximum inconsistency score of partitions n=2, 3, . . . , nₘᵢₙ, and VSₘᵢₙ is the minimum inconsistency score of partitions nₘᵢₙ, and computes p=75^{th} percentile of Sₙ. For example, in the plot 300 of FIG. 3, the cluster module 112 can determine the 75^{th} percentile of Sₙ, of the plot between partition level 1 and partition level 6, which corresponds to point 306. The 75^{th} percentile of Sₙ, here can be a measure of the sharpness or steepness of the curve about the minimum value. However, it should be understood that other steepness or sharpness measures can be employed. After determining the steepness or sharpness measure for the set of features corresponding to the top 1% of features having the highest variance, the method can proceed to step 209 to select another set of features. For example, at step 209, the cluster module 112 can select the set of features corresponding to the top 5% of features having the highest variance, and steps 210-219 can be applied to this set of features, as discussed above. The loop defined by steps 209-220 can proceed to evaluate the sets of features corresponding to the top 10%, the top 15%, etc. of features having the highest variance. In accordance with one exemplary embodiment, the threshold can be set to 15%, where, at step 220, the cluster module 112 can determine that an optimum feature set has been found by determining that all of the sets of features corresponding to the top 1%, 5%, 10% and 15% of the features having the highest variance have been evaluated. Here, at step 220, the cluster module 112 can determine which of the feature sets have a steepness or sharpness measure with the highest magnitude. In addition, the cluster module 112 can select the feature set having the highest magnitude steepness or sharpness measure as the optimum feature set. It should be noted that the evaluation of the optimality of the feature sets need not be performed by increments of 4% or 5%, but can be performed using other percentages or differentiating parameters as a matter of design choice depending on the type of biological data assessed. In addition, the threshold need not be set to 15%, but can also be selected as a matter of design choice. Further, in accordance with one exemplary embodiment, at step 209, prior to selection of any feature sets for evaluation, the cluster module 112 can remove from consideration features corresponding to the top 0.01% of the features having the highest variance as outliers. The outlier threshold, which is in this example, the top 0.01%, can also be selected as a matter of design choice depending on the type of biological data assessed. In response to the cluster module determining that an optimum feature set has been found at step 220, the method can proceed to step 221.

At step 221, the partition evaluation module 114 can identify the subpopulations of the constituents of the biological organism(s) by selecting the partition having the minimum inconsistency score as the subpopulations. Here, the minimum inconsistency score can be the minimum value determined at step 219. If optional steps 209 and 220 are performed, then the minimum inconsistency score used to identify the subpopulations is the minimum inconsistency score obtained for the optimum feature set selected at step 220.

Optionally, at step 222, the clinical data mapper 122 can map and associate clinical data with at least a subset or all of the clusters of the selected partition, and/or assign labels and/or annotations to the subset or all of the clusters of the selected partition. For example, the annotations can include at least one of drug response data, risk of recurrence of a disease (e.g., low risk, medium risk, high risk, etc.) or disease subtype data. Annotations/labels can be received from a user through the user interface 102, stored in the storage medium 108, and correlated to the constituents of the cohort at step 202, discussed above. Based on the correlation, the clinical data mapper 122 can map the annotations/labels to the respective clusters. If the annotations/labels are not available, then the clinical data mapper can generate and assign annotations/labels to each cluster or each cluster in the subset. For example, the annotations/labels can be accessed from an outside database storing information about the biological data clustered in accordance with the method 200. The annotations/labels can indicate which patients received a certain drug, which patients responded well to the drug and which patients did not respond well to the drug to enable the health care practitioner determine whether the drug was effective. Thus, if cluster representations indicate that the patient responded to the drug, then they can indicate to a health care practitioner that the treatment should continue. The annotations/labels can also include clinical or phenotypic data, including subtype data, which in turn can include specific types of cancer that are clinically relevant.

It should be understood that the annotations can include clinical variables, clinical outcomes and/or other clinical labels. For example, in accordance with exemplary aspects, at step 222, each cluster or each cluster of the subset can be assigned or associated with one or more clinical variables, clinical outcomes and/or other clinical labels. For example, a clinical variable can be one or more drugs administered to patients whose biological data was input at step 202, a prescribed diet followed by the patients, and/or a physical therapy regimen undergone by the patients, among other variables. The clinical variable can also include a disease or ailment that the drug, diet and/or physical therapy aimed to cure. In turn, the corresponding clinical outcome can be an indication of whether the drug, diet, or physical therapy resulted in curing or improving the disease or ailment suffered by the patient. The clinical variables and clinical outcomes can be known a priori, before the partition obtaining step 210 is performed. Here, at step 222, by referencing the correlation between patients/biological data and clinical variables and clinical outcomes, the clinical data mapper 122 can map the corresponding clinical variables/clinical outcomes to the clusters/subpopulations identified at step 221. For example, the biological data samples and the values of a centroid, or other mathematical representation, for a cluster/subpopulation determined at step 221, can be mapped to the corresponding clinical variables and clinical outcomes of patients belonging to the respective cluster/subpopulation. For example, the representation generator 124 can formulate the representation as a matrix of proteins sets and/or gene sets and corresponding values denoting, for example, copy number alteration data, gene expression data, and/or methylation data for the set that form a centroid of the biological data of the constituent members of the corresponding cluster determined at step 221. Here, the centroid representation, or other representation, along with the clinical variable/outcome annotations can serve as a model that can act as a guide for the clinical management of new patients, as discussed herein below. It should be noted that any type of annotation/label can be mapped to the respective cluster. For example, besides clinical variables and outcomes, the annotations/labels can be, for example, cancer subtype data. For example, like the clinical variables and outcomes, the clinical labels can be known a priori, before the partition obtaining step 210 is performed. At step 222, by referencing the correlation between patients/biological data and clinical labels, the clinical data mapper 122 can map the corresponding clinical labels to the clusters identified as part of the selected partition at step 221. For example, similar to the clinical variables and outcomes, the biological data samples and the values of the centroid, or other mathematical representation, for a cluster/subpopulation determined at step 221 can be mapped to the corresponding label/subtype of patients belonging to the respective cluster. The centroid/mathematical representation together with the clinical labels can be employed as a model for comparison purposes that can aid in the diagnosis of a patient. The labels can be any clinically relevant data including, for example, recurrence information, survival rate, mutation data for a specific gene or set of genes, and/or expression level of a gene or expression levels of genes of a specific pathway, etc.

At step 224, the representation generator 124 can generate representations of the clusters of the partition selected at step 221 having the minimum inconsistency score and/or representations of the corresponding biological data, including any data labels or annotations mapped or assigned at step 222, and store the generated representations within the storage medium 108. For example, each cluster representation can be a centroid, or another adequate representation, as discussed above with respect to step 222, for a cluster of the selected partition. Alternatively, the representation of a cluster can be a combination of the centroid, or other adequate mathematical representation, and clinical variable/outcome data, clinical label, and/or other annotation mapped to the cluster at step 222. For example, one cluster representation can include a centroid of a cluster, an indication of the drug administered to patients belonging to the cluster, the disease or ailment that the drug treatment aimed to cure and an indication that the drug was successful. Similarly, another cluster representation can include a centroid of a different cluster, an indication of the same drug administered to patients belonging to the cluster, the corresponding disease or ailment and an indication that the drug was not successful. As discussed herein below, the cluster representations can serve as a model that can aid a healthcare provider in assessing whether a new patient will respond well to the drug. Alternatively, a cluster representation can include a centroid of a cluster and a cancer subtype label. Here, the cluster representation can be employed for comparison purposes to aid a health care provider in diagnosing the illness from which a patient is suffering. The computed patterns/representations are visualized and provided with clinical annotation for interpretation. For example, the representation can be a graph, heat map, or 2D plots, where points represent patients or other types of constituents. Further, the representation can include representations of the sets genes or proteins denoting the clusters of the partition selected at step 221.

At step 226, the representation generator 124 can direct the display/output device 104 to display or output the generated representations. As noted above, the representation can be a representation of at least one of the clusters of the partition selected at step 221 or the sets genes or proteins denoting these clusters. Further, at least one of clinical or phenotypic annotations to the clusters can also be displayed. In addition, the identified subpopulations, or, equivalently, the clusters of the partition selected at step 221, can be a simple listing of the identified subpopulations of the constituents. Alternatively, the output of the identified subpopulations can further include statistical characteristics, such as, for example, descriptive characteristics of inter-subpopulation similarities and/or inter-population dissimilarities.

Referring to FIG. 4, with continuing reference to FIGS. 1 and 2, a method 400 for providing diagnostic information in accordance with exemplary embodiments is illustratively depicted. It should be noted that the method 400 can be combined with the method 200. Further, the method 400 can be performed to inform a health care provider of the particular feature data that should be compiled to obtain diagnostic information and, additionally or alternatively, can be performed to provide the diagnostic information to a health care provider. For example, the method 400 can begin at optional step 402 at which the system pre-processor 110 can optionally receive search criteria from a user through the user-interface 102 and can store the criteria in the storage medium 108. For example, the search criteria can denote a particular disease or subtype and/or a particular drug or other treatment that a health care provider is considering to prescribe to a patient. The search criteria received at step 402 can be entered alone or, alternatively, the search criteria can be entered with biological data. For example, at optional step 404, the pre-processor 110 can receive at least one other biological data sample from a user through the user-interface 102 and store the sample in the storage medium 108. The biological data sample can be biological data formulated as a matrix, as discussed above with respect to step 202, and can be composed of an entire genome of a patient or constituent, or can be composed of a subset of genes and/or any set or subset of proteomic data discussed above. Step 404 can also be performed with or without step 402. For example, if both step 402 and step 404 are performed such that the pre-processor 110 receives both search criteria and biological data sample(s), the pre-processor 110 can associate the criteria with the biological data and can store the criteria and biological data in the storage medium 108. Providing search criteria with the data samples can restrict a search for diagnostic information to a specific type of clinical variable, outcome or label. For example, the search can be restricted to diagnostic information related to a particular subtype and/or drug. Alternatively, step 404 can be performed without step 402 to enable a health care provider to obtain all information that is relevant to the biological data sample submitted to the system 106.

At step 406, the diagnostic matcher 126 can retrieve the search criteria and/or biological data sample(s) from the storage medium 108 and can search for one or more matches to the search criteria and/or biological data sample(s) within a database stored in storage medium 108. For example, at step 408, the diagnostic matcher 126 can compare the search criteria to annotations stored in the storage medium 108. For example, the diagnostic matcher 126 can compare the search criteria to clinical variables, clinical outcomes and/or clinical labels stored and associated with cluster representations in a database in storage medium 108. The clinical variables, clinical outcomes and/or clinical labels stored and associated with cluster representations can be the annotations mapped at step 222. Similarly, the diagnostic matcher 126 can compare the biological data received at step 404 to centroids or other mathematical representations or models generated at step 224 of the method 200 and stored in the database in storage medium 108. In addition, if search criteria and biological data are received at steps 402 and 404, the diagnostic matcher 126 can filter the cluster representations and search through only cluster representations that are associated with the search criteria.

At step 410, the diagnostic matcher 126 can determine whether any match or matches to the search criteria and/or the biological data sample(s) is/are found. Here, any of a variety of semantic-based search methods can be employed to implement the searching and matching at steps 406 and 410 with respect to the search criteria. Similarly, the biological data sample(s) can be matched to cluster representations by selecting a cluster representation that falls within a similarity threshold distance, which can be preset or can be set by search criteria received at step 402. For example, the diagnostic matcher 126 can, at comparison step 408, determine a Euclidean distance measure, a Manhattan distance measure, and/or some other appropriate measure, between the biological data sample received at step 404 and all of the cluster representations or a subset of the cluster representations stored in the storage medium 108, where the subset of cluster representations can be determined by filtering the representations with the search criteria, as discussed above. In response to determining that the biological data sample received at step 404 falls within a threshold distance to any one or more cluster representations, which can be centroids, as discussed above, the diagnostic matcher 126 determines that this or these representation(s) are matches to the biological data. Otherwise, in response to determining that the biological data sample received at step 404 does not fall within the threshold distance to any of the stored representations, the diagnostic matcher 126 determines that a match was not found.

If a match to the search criteria and/or the biological data was not found, then the method can proceed to step 414, at which the diagnostic matcher 126 can indicate through the display/output device 104 that no matches were found. Thus, the diagnostic matcher 126 can indicate that the database stored in storage medium 108 lacks particular diagnostic information and can prompt the user to run the method 200 with additional biological data to expand the database.

If a match to the search criteria and/or the biological data was found, then the method can proceed to step 412, at which the diagnostic matcher 126 can output representation(s)/model(s) matched to biological data sample(s) received at step 404, annotations, which can include, for example, clinical variables, clinical outcomes and/or clinical labels, matched to search criteria, and/or diagnostic information associated with the matched representations/model(s). For example, if, at step 402, a user enters search criteria, which can denote, for example, a particular disease or subtype and/or a particular drug or other treatment that a health care provider is considering to prescribe to a patient, without a biological data sample, then the diagnostic matcher 126 can output, through the display/output device 104, the feature data set and/or cluster representation, such as, for example, a centroid, associated with the matched annotations. For example, if the user enters a particular cancer subtype, then the diagnostic matcher 126 can output a gene set and/or a protein set and corresponding genomic/proteomic information associated with the gene set/protein set in the matched representation, such as, for example, copy number variation data, gene expression data and/or gene methylation data. The output can inform the health care provider of the particular biological data that he or she should obtain to determine, using system 106, whether a patient has the subtype in the search criteria. In addition, if a health care provider enters one or more biological data samples and one or more cluster representation matches are found, the diagnostic matcher 126 can output a cancer subtype associated with the matched representation to inform the healthcare provider that the patient likely suffers from this particular subtype. Accordingly, in this way, for example, the system can help guide the clinical management of the patient. Further, if a health care provider enters one or more biological data samples and one or more cluster representations matches are found, the diagnostic matcher 126 can alternatively or additionally, output the clinical variables, such as drug treatments or other types of treatments, and the clinical outcomes associated with the matched representation(s). For example, the diagnostic matcher 126 can in this way notify the health care provider that previous patients in a cluster matching the health care provider's current patient were cured by or responsive to, or were not responsive to, a particular drug therapy. As such, the methods 200, 400 and system 106 can provide effective clinical guidance to the health care provider during therapy planning for the patient.

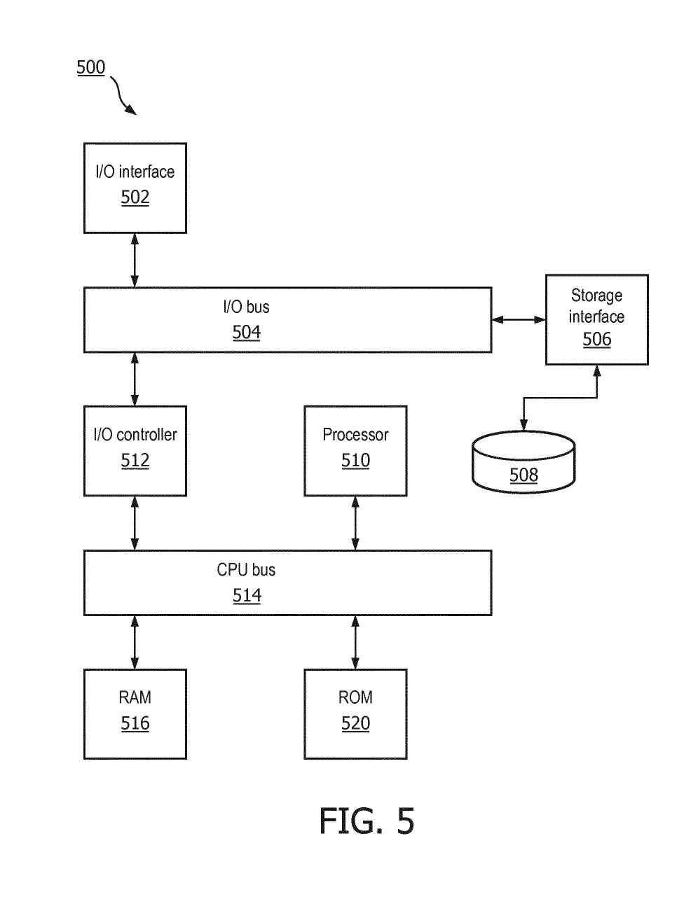

Referring now to FIG. 5, an exemplary computing system 500 by which method embodiments of the present principles described above can be implemented, is illustrated. The computing system 500 includes a hardware processor or controller 510 and a storage medium 508. The processor 510 can access random access memory (RAM) 516 and read only memory (ROM) 520 through a central processing unit (CPU) bus 514. In addition, the processor 510 can also access the computer-readable storage medium 508 through an input/output controller 512, an input/output bus 504 and a storage interface 506, as illustrated in FIG. 5. The processor 510 can implement any one or more of elements 110, 126, 112, 114, 122 or 124. The system 500 can also include an input/output interface 502, which can be coupled to a display/output device 104, the user-interface 102, a keyboard, a mouse, a touch screen, external drives or storage mediums, etc., for the input and output of data to and from the system 500. In accordance with one exemplary embodiment, the processor 510 can access software instructions stored in the storage medium 508 and can access memories 516 and 520 to run the software instructions stored on the storage medium 508. In particular, the software instructions can implement or be the steps of the method 200 and/or method 400. Alternatively, the software instructions that implement method 200 and/or 400 can be encoded in a computer-readable signal medium, such as a radio frequency signal, an electrical signal or an optical signal.

It will be apparent that various alternative hardware to the example computing system 500 may be used to implement the methods and systems described herein. For example, in some embodiments, one or more virtual machines hosted in a cloud computing environment may provide some or all of the functionalities described herein. As such, some of the components of the system 500 may be resident in separate physical devices from each other but, nonetheless, operate together as a single virtual device or grouping thereof. Various modifications to the system to support such an arrangement will be apparent

As discussed above, the bioinformatics methods and systems described herein provide an efficient and accurate means for identifying subpopulations by allocating a non-zero intra-cluster inconsistency measure to one-sample clusters. The embodiments described herein can be employed in any appropriate field utilizing bioinformatics technology. For example, as noted above, embodiments can be employed in clinical applications for purposes of detecting patterns of clonal evolution and tumor heterogeneity to determine aggressiveness of the tumor. In addition, as noted above, embodiments can be used in discovering new population outgrowth in bacterial infections, as well as in other applications. Further, the embodiments can be utilized in therapy design. For example, as noted above, the identification of subpopulations can enable health care professionals to tailor drugs to each subpopulation, thereby significantly enhancing the chances of success of the treatment.

While several embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, materials, and configurations described herein are meant to be exemplary and that the actual parameters, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A system for characterizing patient data for clinical outcome prediction and subtyping by detecting subpopulations of constituents of at least one biological organism comprising:
at least one hardware processor configured to receive biological data samples of said constituents and perform a clustering procedure to obtain a plurality of partitions of the biological data samples of said constituents of said at least one biological organism, each partition of the plurality of partitions defining a respective number of clusters of the biological data samples of said constituents; and
a non-transitory storage medium configured to store the plurality of partitions,
wherein the at least one hardware processor is further configured to compute, for each partition of said plurality of partitions, an inconsistency score for the corresponding partition computed using an inconsistency measure which is a statistical variance measure that measures intra-cluster inconsistency, wherein, for at least one of said plurality of partitions, a non-zero value is allocated to the inconsistency measure of at least one cluster that has only one biological data sample, and wherein the partition evaluation module is further configured to determine which partition of the plurality of partitions has a minimum inconsistency score and to identify said subpopulations of said constituents of the at least one biological organism by selecting the partition of the plurality of partitions having the minimum inconsistency score as said subpopulations.

2. The system of claim 1, wherein the at least one hardware processor is further configured to weight the inconsistency measure of each cluster of at least a subset of clusters in the corresponding partition as a function of a total number of biological data samples in the corresponding cluster and of a total number of biological data samples of the constituents of the at least one biological organism.

3. The system of claim 1, wherein at least one hardware processor is configured to determine the non-zero value by weighting an inconsistency measure of the biological data samples of said constituents of said at least one biological organism as a whole.

4. A method for characterizing patient data for clinical outcome prediction and subtyping by detecting subpopulations of constituents of at least one biological organism, said method being implemented by at least one hardware processor and comprising:
Receiving biological data samples of said constituents and performing a clustering procedure to obtain a plurality of partitions of the biological data samples of said constituents of said at least one biological organism, each partition of the plurality of partitions defining a respective number of clusters of the biological data samples of said constituents;
for each partition of said plurality of partitions, computing an inconsistency score for the corresponding partition computed using an inconsistency measure which is a statistical variance measure that measures intra-cluster inconsistency, wherein, for at least one of said plurality of partitions, a non-zero value is allocated to the inconsistency measure of at least one cluster that has only one biological data sample;
determining which partition of the plurality of partitions has a minimum inconsistency score; and
identifying said subpopulations of said constituents of the at least one biological organism by selecting the partition of the plurality of partitions having the minimum inconsistency score as said subpopulations.

5. The method of claim 4, wherein the biological data samples includes at least one of genomic data or proteomic data.

6. The method of claim 4, wherein the computing further comprises weighting the inconsistency measure of each cluster of at least a subset of clusters in the corresponding partition as a function of a total number of biological data samples in the corresponding cluster and of a total number of biological data samples of the constituents of the at least one biological organism.

7. The method of claim 6, wherein the weighting is performed such that the inconsistency measure of the corresponding cluster of the at least the subset of clusters is directly related to the total number of biological data samples in the corresponding cluster.

8. The method of claim 4, wherein the non-zero value is determined by weighting the inconsistency measure of the biological data samples of said constituents of said at least one biological organism as a whole.

9. The method of claim 8, wherein the weighting comprises weighting the inconsistency measure of the biological data samples of said constituents with a total number of biological data samples of the constituents of the at least one biological organism.

10. The method of claim 9, wherein the weighting is performed such that the non-zero value is inversely related to the total number of biological data samples of the constituents of the at least one biological organism.

11. The method of claim 4, wherein the inconsistency measure is a statistical variance of pairwise distances between biological data samples in a given cluster of the corresponding partition.

12. The method of claim 4, further comprising: displaying a representation of at least one cluster of the selected partition, wherein said displaying comprises displaying at least one of clinical or phenotypic annotations to said at least one cluster of the selected partition.

13. The method of claim 12, wherein said annotations include at least one of drug response data, risk of recurrence of a disease or disease subtype data.

14. The method of claim 4, further comprising:
associating at least a subset of the clusters of the selected partition with at least one of clinical variables, clinical outcomes or clinical labels;
receiving at least one other biological data sample as a query;
searching for at least one match to said at least one other biological data sample by comparing the at least one other biological data sample to representations of clusters of the selected partition; and
outputting the at least one of clinical variables, clinical outcomes or clinical labels associated with a representation of at least one of the clusters of the selected partition matching said at least one other biological data sample as diagnostic information.

15. A computer-readable medium comprising a computer-readable program that, when executed on a computer, enables the computer to perform the method of claim 4.
